(19) United States
(12) Patent Application Publication
(10) Pub. No.: US 2007/0264191 A1
(43) Pub. Date: Nov. 15, 2007

(54) Materials and Methods Relating to the Treatment of Glioblastomas

(76) Inventors: Ivy A.W. Ho, Singapore (SG); Paula Y.P. Lam, Singapore (SG); Kam M. Hui, Singapore (SG)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 10/592,433

(22) PCT Filed: Mar. 11, 2005
(86) PCT No.: PCT/GB05/01015

§ 371 Date: Jul. 18, 2007

(30) Foreign Application Priority Data
Mar. 11, 2004 (GB) 0405482.1

(51) Int. Cl. A61K 38/10; A61K 35/76; A61K 38/16; A61K 51/08; A61P 35/00; C07K 7/08; C12Q 1/16; G01N 33/574

(52) U.S. Cl. 424/1.69; 424/780; 435/35; 435/7.23; 514/12; 514/14; 530/327

(57) ABSTRACT

The invention provides human glioma-specific peptides as delivery vehicles for therapeutic agents. The peptides may be complexed with a diagnostic or therapeutic agent.

### FIELD OF THE INVENTION

The present invention concerns materials and methods relating to the treatment and diagnosis of glioblastomas. Particularly, but not exclusively, the invention provides human glioma-specific peptides as delivery vehicles, for substances such as genes.

### BACKGROUND OF THE INVENTION

Identification of molecules that mediate the targeted delivery of therapeutic DNA or drugs to specific cell types can significantly reduce the amount of therapeutic DNA or drugs needed, and would therefore reduce the potential toxicity to non-targeted cells. Glioblastomas multiforme (GBM) are very malignant tumors and account for over 20% of primary brain tumors in adults. In spite of advances in surgery, chemotherapy, and radiotherapy, the life expectancy of patient with glioblastoma is approximately 11 months (Cowan et al., 2000). A prominent clinical feature of GBM is their ability to infiltrate into surrounding brain tissues, thus render the complete surgical removal practically impossible. One way to overcome this difficulty is to design strategies that allow the delivery of therapeutic genes or drugs specifically to cancer cells. Gene therapy could be offered as a viable therapeutic option and many attempts have been exploited.

One of the common methods to enable gene expression in a selected cell population is to incorporate gene expression cassettes under specific promoter to ensure transgene expression in a restricted specific cell types. Various tissue-specific or tumor-specific promoters have been employed to restrict transgene expression to particular cell types (Shibata et al., 2000; Ueno et al., 2001). However, tissue specific promoter generally suffers from weak induction of transgene expression, and rely on inducible agents that might exert unfavorable effects on the host (Agha-Mohammadi and Lotze, 2000; Nettelbeck et al., 2000). Moreover, the essential upstream control region that governs gene expression from a particular promoter can be widely distributed, this represents a major hindrance in the construction of the transcriptionally-mediated targeted vector systems (Agha-Mohammadi and Lotze, 2000).

There has been much progress in the field of vector targeting using different vector systems (Ohno et al., 1997; Laquerre et al., 1998; Frederiksen et al., 2000; Reddy et al., 2002). Wu et al (1987) is the first to report the use of the glycoprotein asialoorosomucoid (ASOR) to specifically target the liver parenchyma (Wu and Wu, 1987). Retroviral vectors have recently been modified to contain a chimeric envelope and thus allowed targeting to breast cancer cells (Tai et al., 2003). Around 50% of GBM shows amplifications or mutations that activate gene encoding the epidermal growth factor (EGF) receptor, thus producing a constitutively active receptor in the absence of the EGF ligand. (Mischel and Cloughesy, 2003). These characteristics of glioma cells have been exploited in designing vectors that deliver therapeutic agents to tumor cells that over-expresses this growth factor receptor. Doubly ablated adenoviral vectors, lacking both the coxsackievirus-adenovirus (CAR) receptor and the alpha(v) integrin binding capacities, together with bispecific single-chain antibodies that recognize both the human epidermal growth factor receptor (EGFR) or the epithelial cell adhesion molecule have been employed for specific gene delivery to primary human brain tumors (van Beusechem et al., 2002; Samoylova et al., 2003). Fusion proteins consisting or peptide toxins fused to the human EGF (DAB 389EGF) has been demonstrated to selectively kill cells that over-expresses EGFR (Cohen et al., 2003).

Phage display techniques have been used for selection on whole cells to identify peptide ligands directed against particular cell surface protein (Parmley and Smith, 1989; Barry et al., 1996; Szardenings et al., 1997; Campa et al., 2002; Liu et al., 2003). Using this technique, peptides that bind to kidney, lung, skin, pancreas, intestine, uterus, adrenal gland, retina, fibroblast cells, myoblast, myotubes, human neutrophils, human laryngeal carcinoma cells, endothelial cells (Barry et al., 1996; Arap et al., 1998; Koivunen et al., 1999; Pasqualini et al., 2000; White et al., 2001), human colorectal cell line (Rasmussen et al., 2002) have been identified. Recently, peptides that home selectively to the vasculature of various organs were isolated by the in vivo biopanning of peptide phage display libraries in mice (Pasqualini and Ruoslahti, 1996; Rajotte et al., 1998; Arap et al., 2002a; Essler and Ruoslahti, 2002; Trepel et al., 2002), as well as in human (Arap et al., 2002b). The selected sequence can be used to target therapeutic agents (Arap et al., 1998) and diagnostic imaging radiolabels (Schumacher et al., 2002).

Gene transfers to malignant gliomas have been extensively pursued both in cell culture as well as in animal model experiments. However, clinical gene therapy trials for recurrent gliomas did not fulfill the high expectations suggested by laboratory preclinical results. One of the significant pitfall is the low transduction efficiences of tumor cells observed in vivo (Weyerbrock and Oldfield, 1999; Rainov and Kramm, 2001; Rainov and Ren, 2003), resulting in low expression of therapeutic gene products to efficiently eradicate the tumor cells. Retrovirus vector was one of the earliest viral vectors to be employed for the delivery of therapeutic genes to gliomas in the clinical settings (Rainov and Ren, 2003). Although both Phase I and II clinical studies showed favorable safety profile and some efficacy, phase III study failed to demonstrate significant extension of progression-free or overall survival (Rainov and Ren, 2003). The use of adenovirus as well as herpes simplex virus-based vectors have also been actively pursued both in the laboratory and clinical settings (Bansal and Engelhard, 2000). One of the most common strategy employed for gene delivery to the brain involved the direct stereotactic injection to the coordinate of interest. Most often this approach is limited by the volume and number of injections that one would need in order to achieve therapeutic doses. Moreover, with this route of administration, the vector is only taken up by cells in the immediate vicinity of the injection site. Improved strategies for selective delivery of therapeutic agents are therefore needed to combat the devastating and usually fatal cancer.

Sequences that target to human malignant glioma cells have been demonstrated for glioma cell lines (Spear et al., 2001; Zhang et al., 2001). However, their specificity is limited and confined to a single cell line. Spear et. al. reported a peptide that is specific for U87MG glioma cell line and having the sequence, MCPKHPLGC, but did not bind to other glioma cells (Spear et al., 2001). Zhang et. al. reported a sequence that binds to glioma cells. However, this sequence is not specific for gliomas and it also binds to other cancer cell lines (Zhang et al., 2001).

### SUMMARY OF THE INVENTION

At its most general, the present invention provides materials and methods relating to the diagnosis and/or treatment of glioblastomas using peptides capable of specifically binding to glioma cells.

The present inventors have for the first time determined a group of peptides (see Table 1) that bind specifically to a range of glioma cells. By “specific” we mean that they do not bind cancer cells of non-glial origins. Preferably the peptides do not bind normal cells, including normal glial cells.

In a first aspect of the invention there is provided a peptide being no more than 30 amino acids in length and comprising a sequence selected from the group consisting of

| | |
|---|---|
| SGHQLLLNKMPN, | (SEQ ID NO. 1) |
| LWATFPPRPPWL, | (SEQ ID NO. 2) |
| WSAAPTKPPYHT, | (SEQ ID NO. 3) |
| ILANDLTAPGPR, | (SEQ ID NO. 4) |
| HHGHSPTSPQVR, | (SEQ ID NO. 5) |
| LPYGTSWRHAPV, | (SEQ ID NO. 6) |
| YVQGWNYHDLTR, | (SEQ ID NO. 7) |
| LWAAFPPQASVA | (SEQ ID NO. 8) |
| and | |
| FDTPHTLTWFHG | (SEQ ID NO. 9) |

The invention further provides a peptide, the amino acid sequence of which consists of an amino acid sequence selected from the group consisting of

| | |
|---|---|
| SGHQLLLNKMPN, | (SEQ ID NO. 1) |
| LWATFPPRPPWL, | (SEQ ID NO. 2) |
| WSAAPTKPPYHT, | (SEQ ID NO. 3) |
| ILANDLTAPGPR, | (SEQ ID NO. 4) |
| HHGHSPTSPQVR, | (SEQ ID NO. 5) |
| LPYGTSWRHAPV, | (SEQ ID NO. 6) |
| YVQGWNYHDLTR, | (SEQ ID NO. 7) |
| LWAAFPPQASVA | (SEQ ID NO. 8) |
| and | |
| FDTPHTLTWFHG | (SEQ ID NO. 9) |

However, preferably the peptide is up to 30 amino acids in length, more preferably up to 25, up to 20, up to 19, up to 18, up to 17, up to 16, up to 15, up to 14 or up to 13 amino acids in length.

A preferred peptide comprises or consists of SEQ ID NO. 2, designated herein MG11.

It will be appreciated that the sequences identified by the inventors may be modified by one or more amino acids so as to alter their binding affinity to glioma cells. The modification may be by substitution, deletion or addition of one or more amino acids. Preferably, the modification serves to improve the properties of the peptide e.g. binding affinity of the peptide. Modification of the sequences is described in more detail below but, in accordance with this, the present invention further provides a peptide having up to 30 amino acids in length comprising an amino acid sequence having at least 8 residues identical with corresponding residues in an amino acid sequence selected from the group consisting of

| | |
|---|---|
| SGHQLLLNKMPN, | (SEQ ID NO. 1) |
| LWATFPPRPPWL, | (SEQ ID NO. 2) |
| WSAAPTKPPYHT, | (SEQ ID NO. 3) |
| ILANDLTAPGPR, | (SEQ ID NO. 4) |
| HHGHSPTSPQVR, | (SEQ ID NO. 5) |
| LPYGTSWRHAPV, | (SEQ ID NO. 6) |
| YVQGWNYHDLTR, | (SEQ ID NO. 7) |
| LWAAFPPQASVA | (SEQ ID NO. 8) |
| and | |
| FDTPHTLTWFHG | (SEQ ID NO. 9) |

wherein the peptide is capable of binding glioma cells.

Preferred sizes of the peptide are stated previously. Peptides having at least 8 residues identical with corresponding residues of SEQ ID NO. 2 are preferred.

More preferably, the minimum number of identical residues is 9, 10 or 11.

Owing to the abilities of the peptides of the invention to specifically target glioma cells, the inventors have appreciated that they will provide useful tools in targeting therapeutic and diagnostic agents to glioma cells.

Thus in further aspects of the invention there is provided a complex comprising a peptide of the invention associated with (e.g. covalently or non-covalently linked to) a therapeutic agent or a diagnostic agent.

A complex comprising the peptide of the invention and a diagnostic agent may be used in in vitro or in vivo methods of diagnosing glioblastomas. The diagnostic agent may be a label, e.g. a radioactive, fluorescent, chemiluminescent or enzyme label. Suitable fluorescent labels include fluorescent proteins such as green fluorescent protein. Enzyme labels typically act on a substrate to produce a detectable change, e.g. a spectrophotometrically detectable change. Examples are well known to the skilled person and include luciferase, alkaline phosphatase and horseradish peroxidase.

The therapeutic agent may be a protein (e.g. an enzyme), a peptide, or a drug (e.g. a cytotoxic agent). Suitable enzymes include those capable of converting prodrugs to drug molecules (see below). Examples of cytotoxic agents include 6-Diazo-5-oxo-L-norleucine [DON], Actinomycin D, Mitomycin, Mitoxantrone, Cisplatin, Melphalan, Etoposid, 5FU, antracyclines, Paclitaxel and Tamoxifen. The therapeutic agent may also be a chemokine or an immune-enhanced, e.g. melatonin.

The therapeutic or diagnostic agent may be a nucleic acid molecule, e.g. DNA, RNA, cDNA or the like. For diagnostic purposes, the nucleic acid may encode a label, such as a fluorescent protein or enzyme label as described above.

For therapeutic purposes, the nucleic acid may encode a protein which has the ability to kill the cell in which it is expressed. For example, the nucleic acid may encode an apoptosis agent. Alternatively, the nucleic acid may encode an enzyme capable of converting prodrugs to drug molecules.

In either case, the nucleic acid molecule may be part of an expression vector. The vector may be capable of becoming incorporated into the genome of the glioma cell such that it can be expressed, or may be an episomal element (e.g. a plasmid) which allows expression without incorporation into the cell genome.

In order to effectively deliver nucleic acid into the glioma cells and to ensure uptake, intracellular trafficking and nuclear retention (where appropriate), other components may be used. These may include cationic lipids, polymers, viral vectors, etc.

Thus the expression vector may be a viral vector, in which case the complex of peptide and associated nucleic acid may be a viral particle. The vector may be, for example, a retroviral or adenoviral vector. The peptide will typically be displayed upon the surface of the viral particle to allow targeting of the virus to the glioma cell. For example, the peptide may be expressed as part of a viral coat protein.

Alternatively the complex may comprise a polycation (e.g. polylysine) to facilitate association between the peptide of the invention and a nucleic acid, via electrostatic interactions between the polycation and the negatively charged nucleic acid backbone. The polycation may be covalently linked to the peptide.

As mentioned above, the invention also provides methods of treating glioma using genetically directed enzyme prodrug therapy, where the promoter and enzyme prodrug system are directed to the glioma cells using a peptide of the invention.

Genetically directed enzyme prodrug therapy, (GDEPT), comprises two parts: a tumour specific promoter and the enzyme prodrug system. The properties of an optimal tumour selective transcriptional activation system can be summarized as, ideally tumour specific, only expressed in nonessential tissues, no cross-specificity with unusual but essential cell types, regulatory elements from gene cloned and sequenced, specific transcription factor binding sites identified, enhancer and inhibitory factors understood.

The present invention therefore provides the use of nucleic acid molecules comprising promoters to drive expression of enzyme-prodrug activation systems such as viral thymidine kinase and Gancyclovir, although many other systems known to those skilled in the art may also used. Targeted gene expression via an appropriate gene promoter may also be used in gene replacement strategies for cancer therapy. The present invention provides a system for use in the control of neoplasia in a human or animal subject comprising a vector or other delivery system comprising a peptide of the invention capable of selectively infecting glioma cells in said subject, said vector carrying a DNA or RNA sequence encoding an enzyme operably linked to a promoter sequence of the present invention, in association with a prodrug capable of being converted into an active compound by the action of said enzyme.

The invention also provides a pharmaceutical composition comprising the complex of the invention along with a pharmaceutical acceptable carrier.

The use of the peptides to target nucleic acid molecules, drugs, etc. directly to glioblastomas means that the doses of the relevant therapeutic or diagnostic agents may be reduced and consequently any potential side-effects resulting from these agents may also be reduced.

The invention further provides pharmaceutical compositions comprising the peptide of the invention and the therapeutic or diagnostic agent. Compositions comprising nucleic acid therapeutic agents may be used in a method of gene therapy. It is further preferably that the pharmaceutical comprises a pharmaceutically acceptable carrier, e.g. excipients, buffers, preservatives and stabilizers.

The invention also provides a method of producing a pharmaceutical or diagnostic composition as previously defined, comprising admixing a peptide complex (a peptide of the invention associated with a therapeutic or diagnostic agent) together with one or more pharmaceutically acceptable ingredients, i.e. a carrier, stabilizer etc.

As a further aspect of the present invention, there is provided a method of diagnosing a glioblastoma in an individual, said method comprising

a) contacting said brain tissue cells (in vivo or in vitro) with a peptide in accordance with the first aspect of the present invention, wherein said peptide is associated with a diagnostic agent;

b) detecting the presence of the diagnostic agent; and

c) determining the presence of glioma cells.

The method preferably uses a brain tissue sample that has been previously obtained.

As described above, the diagnostic agent may be a label may be a chemical label, e.g. a fluorescent or radioactive label, or it may be a peptide label which can be detected by a specific antibody, or it may be a nucleic acid encoding a protein or enzyme label.

The method of diagnosis may take place in vivo. For example, the complex comprising the peptide and the diagnostic agent may be administered to the individual, e.g. by injection, and the presence of the diagnostic agent detected.

In a further aspect of the present invention there is provided a method of treating an individual having or suspected of having a glioblastoma, said method comprising administering to said individual a peptide in accordance with the first aspect of the invention associated with a therapeutic agent. Administration of the peptide complex (peptide and therapeutic agent) will preferably be by oral administration or injection.

The present invention further provides a peptide or peptide complex in accordance with the earlier aspects of the invention for use in a method of medical treatment.

The invention further comprises the use of a peptide or peptide complex in accordance with the earlier aspects of the invention in the preparation of a medicament for treating glioblastoma. The peptide is preferably associated with a therapeutic agent.

The invention further comprises the use of a peptide or peptide complex in accordance with the earlier aspects of the invention in the preparation of a composition for the diagnosis of glioblastoma. The peptide is preferably associated with a diagnostic agent.

The medicament or diagnostic composition may further comprise cationic lipids, vectors, e.g. viral vectors, or polymers to aid in the stability of the peptide and/or the therapeutic or diagnostic agent.

The present invention provides, as a further aspect, a method of improving the binding affinity of the peptides in accordance with the first aspect of the invention. The method may include modifying the amino acid sequence of the peptide either by addition, deletion or substitution of amino acid residues and then testing said modified peptide for improved binding affinity with glioma cells. The testing step may also include the unmodified peptide as a control so as to compare binding affinities.

Similarly, the invention also provides a method of designing a mimetic of a peptide of the invention, the mimetic being capable of binding glioma cells, said method comprising
a) analysing a peptide of the invention that is capable of binding glioma cells to determine the amino acid residues essential and important for the activity to define a pharmacophore; and
b) modelling the pharmacophore to design and/or screen candidate mimetics having the biological activity, i.e. ability to bind glioma cells.

Preferably, the method includes a step of assaying binding of a candidate mimetic to glioma cells in vitro. Having identified a candidate mimetic that is capable of such in vitro binding, it is preferably optimised for in vivo use. Following said optimisation, the optimised mimetic may be used in any aspect of the present invention.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1. In vitro specificity of MG11 phage to a panel of human glioma cell lines.

Tumor cell lines of various origins were incubated with 10¹² pfu/μl of MG11 at 37° C. for 2 hours. Mean value for phage recovered from the binding assay and the SEM from triplicate experiments were shown.

FIG. 2. In vivo targeting of MG11 phage to tumor cells of glioma origins.

(A) Binding of MG11 phage to different tissues. (B) Specific binding of MG11 phage to dGli36(i) or SF767(ii) human glioma xenograft as compared to a non-glioma xenograft, CNE2. Bars showed SEM of 3 mice. (C) Targeting of MG11 phage to intracranial dGli36 human glioma xenograft as compared to the normal region of the brain. Data shown are averages of 4 mice. *p≦0.05 (paired t-test).

FIG. 3. In vitro binding of (K₁₆)-MG11 peptide to glioma cells.

(A) Proposed interaction of (K₁₆)-MG11(i) and (K₁₆)-H42(ii) peptide sequence with DNA and their effects on luciferase transgene expression. 3×10⁵ cells were transfected with peptide: DNA ratio of 1:2, 2:1, 10:1 and 20:1, for 24 h. Mean value for luciferase activities and SEM from triplicate experiments were shown. (B) Specificity of (K₁₆)-MG11 peptide on various tumor cell lines. (i) Peptide/DNA (2:1 weight: weight ratio) complexes prepared from (K₁₆)-MG11 was used to transfect pNGVL-Luc into various tumor cell lines. Luciferase activity was analyzed 24 h and 48 h post transfection. (ii) 2:1 weight: weight ratio of peptide: DNA complexes prepared from (K₁₆)-MG11 was used to transfect pEGFP-N1 into U251MG human glioma cells and HepG2 hepatoma cells. Fluorescence pictures were taken 48 h post transfection. (C) Specificity of (K₁₆)-MG11 when compared with (K₁₆)-H42 control peptide. Peptide/DNA complexes prepared from (K₁₆)-MG11 and (K₁₆)-H42 were complexed with pNGVL-Luc and transfected into SF767 and CNE2 tumor cell lines. Luciferase activity was analyzed 48 h post transfection. The luciferase activities shown were SEM of triplicates.

FIG. 4. In vitro specificity of the Lissamine-rhodamine labeled (K₁₆)-MG11 peptide.

100 ng of (K₁₆)-MG11 were incubated with various tumor cell lines for 20 min at 37° C. Cells were subsequently fixed and mounted as described herein. Fluorescence images were studied using LSM 510 confocal microscopy system.

FIG. 5. Binding of Lissamine-rhodamine labeled (K₁₆)-MG11 peptide to primary human glioma culture.

Primary human glioma culture (A), SF767 human glioma cells (B), and normal human astrocytes (C) were incubated with 100 ng of (K₁₆)-MG11 for 20 min at 37° C. Cells were subsequently fixed and mounted as described in Materials and Methods. Fluorescence images were studied using the LSM 510 Meta confocal microscopy system.

FIG. 6. In vivo targeting of the (K₁₆)-MG11 fluorescent-labelled peptide.

SF767 human glioma cells were inoculated into the right flank of immunodeficient mice. 100 μg of lissamine-rhodamine (K₁₆)-MG11 peptide (A) or (K₁₆)-H42 peptide (B) were injected intratumorally into separate mice bearing the SF767 human glioma xenograft and allowed to circulate for 20 min. After which, the tumors were harvested and cryosectioned. Fluorescence images were analyzed using the LSM 510 Meta confocal microscopy system. “I” denotes insert.

### DETAILED DESCRIPTION

Peptides

The term ‘peptide’ is intended to refer to a molecule consisting of several amino acids, adjacent pairs of amino acids being linked together by peptide bonds. The peptides of the invention may be modified by deletion, addition and/or substitution of other amino acid residues and yet maintain and/or improve their biological activity, i.e. ability to bind glioma cells.

Conservative substitution may be defined as a substitution within an amino acid class and/or a substitution that scores positive in the BLOSUM62 matrix.

According to one classification, the amino acid classes are acidic, basic, uncharged polar and nonpolar, wherein acidic amino acids are Asp and Glu; basic amino acids are Arg, Lys and His; uncharged polar amino acids are Asn, Gln, Ser, Thr and Tyr; and non-polar amino acids are Ala, Gly, Val, Leu, Ile, Pro, Phe, Met, Trp and Cys.

According to another classification, the amino acid classes are small hydrophilic, acid/acidamide/ hydrophilic, basic, small hydrophobic and aromatic, wherein small hydrophilic amino acids are Ser, Thr, Pro, Ala and Gly; acid/acidamide/hydrophilic amino acids are Asn, Asp, Glu and Gln; basic amino acids are His, Arg and Lys; small hydrophobic amino acids are Met, Ile, Leu and Val; and aromatic amino acids are Phe, Tyr and Trp.

Substitutions which score positive in the BLOSUM62 matrix are as follows:

| | Original residue | | | | | | | | | | | | | | | | | | | |
|---|---|---|---|---|---|---|---|---|---|---|---|---|---|---|---|---|---|---|---|---|
| | C | S | T | P | A | G | N | D | E | Q | H | R | K | M | I | L | V | F | Y | W |
| Substitution | — | T | S | | S | | S | N | D | E | N | Q | E | I | M | M | M | Y | H | F |
| | | A | | | | | D | E | Q | R | Y | K | Q | L | L | I | I | W | F | Y |
| | | N | | | | | H | | K | K | | | R | V | V | V | L | | W | |

Sequence Identity

Percent (%) amino acid sequence identity with respect to a reference sequence is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. % identity values may be determined by WU-BLAST-2 (Altschul et al., Methods in Enzymology, 266:460-480 (1996)). WU-BLAST-2 uses several search parameters, most of which are set to the default values. The adjustable parameters are set with the following values: overlap SPAN=1, overlap fraction=0.125, word threshold (T)=11. A % amino acid sequence identity value is determined by the number of matching identical residues as determined by WU-BLAST-2, divided by the total number of residues of the reference sequence (gaps introduced by WU-BLAST-2 into the reference sequence to maximize the alignment score being ignored), multiplied by 100.

Synthesis of Peptides

Peptides may be generated wholly or partly by chemical synthesis. The peptides of the invention can be readily prepared according to well-established, standard liquid or, preferably, solid-phase peptide synthesis methods, general descriptions of which are broadly available (see, for example, in J. M. Steward and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, N.Y. (1984); and Applied Biosystems 430A Users Manuals, ABI Inc., Foster City, Calif.), or they may be prepared in solution, by the liquid phase method or by any combination of solid-phase liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Mimetics

Non-peptide “small molecules” are often preferred to peptides for in vivo pharmaceutical use. Accordingly, mimetics of the peptides of the invention may be designed for pharmaceutical use.

The designing of mimetics to a known pharmaceutically active compound is a known approach to the development of pharmaceuticals based on a “lead” compound. This might be desirable where the active compound is difficult or expensive to synthesises or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. Alanine scans of peptide are commonly used to refine such peptide motifs. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. Alternatively, where the mimetic is peptide based, further stability can be achieved by cyclising the peptide, increasing its rigidity. The mimetic or mimetics found by this approach can then be screened to see whether they have the target property, or to what extent they exhibit it. Further optimisation or modification can then be carried out to arrive at one or more final mimetics for in vivo or clinical testing.

### BACKGROUND OF THE INVENTION

Cell Lines

dGli36 cells (gift from Dr. M Sena-Esteves, Children's Hospital of Philadelphia, Philadelphia, Pa.) which over-expresses a truncated mutant EGFR commonly found in human gliomas, were grown in the presence of 1 μg/ml of puromycin (Sigma-Aldrich Corp., St. Louis, Mo.). S F767 and U251MG human gliomas were kindly provided by Dr. DF Deen (Brain Tumor Research Center, UCSF, School of Medicine, Ca.). CNE 2 cells are derived from undifferentiated human nasopharyngeal carcinoma (gift from Prof. HM Wang, Cancer Institute, Guangzhou, People's Republic cf China). All human tumor cell lines, A549 (lung adenocarcinoma); CNE2 (nasopharyngeal carcinoma); HeLa (cervical carcinoma); HepG2 (hepatoma); HK1 (nasopharyngeal carcinoma); KOSC-3 (head and neck carcinoma); KZ2 (melanoma); M059K (glioblastoma); SF767 (glioblastoma); T98G (glioblastoma); U87MG (glioblastoma); U251MG (glioblastoma); U373MG (anaplastic astrocytoma); WT18 (lymphoma) ; used in this study were maintained in Dulbecco's modified Eagle's medium (DMEM) supplemented with 10% fetal bovine serum (FBS) (Hyclone Laboratories, Logan, Utah), 100 U/ml penicillin (Invitrogen, Grand Island, N.Y.), 100 μg/ml streptomycin (Invitrogen, Grand Island, N.Y.), and 2 mM L-Glutamine (Sigma-Aldrich Corp., St. Louis, Mo.). Normal human astrocytes purchased from Cambrex Bio Science Walkersville, Inc., (Walkersville, Md.) were cultured in Astrocyte Basal Medium supplemented with recombinant human EGF, insulin, ascorbic acid, GA-100, L-glutamine and FBS as recommended by the supplier (Cambrex Bio Science Walkersville, Inc., Walkersville, Md.).

Primary Glioma Cell Culture

A single primary glioma biopsy was kindly provided by Dr. Thomas, J. (Department of Neurosurgery, Singapore General Hospital, Singapore) with patient's consent. Tissues were immediately kept in DMEM with 10% FBS. To obtain single cell suspension, the tissues were digested for 5 min with 0.25% trypsin, followed by washing twice with complete DMEM medium. The cells were plated onto a single well of a 96-well plate (Nunc, Roskilde, Denmark), and incubated at 37° C. in a humidified incubator with 5% CO₂ for 24 h before being assayed for the binding of the MG11 peptide.

Phage-Display Library Biopanning

The phage display library employed for biopanning for glioma specific phage was the Ph.D. 12 library (New England Biolabs, Beverly Mass., USA). Phage was selected for binding to human glioma cell line by panning against intact cells in suspension. Glioma cell lines (dGli36, SF767, U87MG, U251, U373MG) were grown in monolayer until confluent, and harvested by treating the cell with phosphate buffered saline (PBS) containing 5 mM EDTA The five glioma cell lines were mixed together in equal proportions to give a final cell number of 1×10⁶. The cell mixture was incubated with 1×10¹¹ pfu of the M13 phage at 37° C. for 2 h. The cells were washed once with PBS containing 0.1% Tween-20 and 9 times with PBS, followed by pelleting and resuspension to remove unbound phages. Bound phages were recovered by eluting with 0.2 M Glycine pH 2.2 containing 1 mg/ml bovine serum albumin (BSA) (Sigma-Aldrich Corp., St. Louis, Mo.), followed by neutralization with 1 M Tris-Cl (pH 9.1). Recovered phages were amplified in ER2537 bacteria (New England Biolabs, USA) and subjected to two addition rounds of enrichment panning. These enrichment panning were then followed by three rounds of subtraction panning against A549, CNE2, and HepG2 successively. The isolated phage clones were titered using ER2537 bacteria. To determine the in vitro specificity of the recovered phages to glioma and non-glioma cell lines, 10¹² pfu of the isolated phage clone was incubated with 1×10⁶ cells at 37° C. for 2 h. Unbound phages were removed and bound phages were recovered and titered as mentioned.

Amplification of Phage Clones

The ER2537 bacteria were employed for phage amplification. To a 20 ml of log phase bacteria culture, 1×10¹⁰ pfu of the phage were added and the mixture was incubated for 4.5 h at 37° C. in a shaking incubator. To precipitate the phages, ⅙ volume of PEG-8000/NaCl solution was added to the bacteria/phage mixture and precipitated at 4° C. overnight. The next day, phages were harvested by centrifugation at 13 000 rpm for 10 min at 4° C. The resulting phage pellet was resuspended in 1 ml Tris Buffered Saline (TBS). The phages were further precipitated by adding ⅙ volume of the PEG/NaCl solution. After incubating for 1 h on ice, the phages were pelleted at 13 000 g for 10 min at 4° C. and resuspended in 200 μTBS containing 0.01% sodium azide. The amplified phages were titered and stored at 4° C.

Titering of Page

To determine the titer, the amplified phages were serially diluted in Luria Broth (LB). Each of the diluted phage solution was added to a log culture of the ER2537 bacteria. After incubating for 5 min at r.t. to allow infection to take place, 3 ml of melted 0.7% agarose (45° C.) were added and the mixture was poured onto a LB agar plate containing 40 mg/ml of 5-bromo-4-chloro-3-indolyl-β-D-galactoside (X-Gal) (Bio-Rad Laboratories, Hercules, USA), and 50 mg/ml isopropyl P-D-thiogalactoside (IPTG) (Invitrogen, Grand Island, N.Y.). The titer of the phage solution was determined by counting the number of blue plaques after 24 h of incubation.

In Vivo Targeting of Phage to Tumor Xenograft

6 week-old female nude mice were purchased from Animal Resource Center (Western Australia). 2×10⁶ dGli36 cells suspended in 10 μl of PBS was stereotactically inoculated into the bregma region (2 mm lateral, 0.25 mm depth) of the right hemisphere of the mice. Tumor growth was monitored by magnetic resonance imaging (MRI), hematoxylin and eosin (H&E) staining, and loss of weight. Phage was injected via tail vein 10 days post-inoculation of tumor when the tumor volume was approximately 75 mm³. For subcutaneous (s.c.) tumor, 5×10⁶ SF767 or dGli36 glioma cells and CNE2 (non-glioma cells control) were suspended in 100 μl of PBS and injected into the right and left flank of 6 weeks old SCID mice (Animal Resource Center, Western Australia), respectively. Tumor growth was monitored by measuring the tumor volume. The tumor volume was calculated using the formula: tumor volume (mm³)=0.52×(width [mm²]×(length [mm]) (Bergers et al., 1999). Phage was injected via tail vein 7 days post-implantation of tumor when the tumor volume was approximately 100 mm³.

Tumor bearing mice were randomized into two groups (5 per group), namely the control group and the experimental group. 1×10¹² plaque forming units of either the control phage or the phage bearing the MG11 sequence suspended in 400 μl of DMEM, was injected via tail vein into tumor bearing mice, and allowed to circulate for 24 h. After 24 h, mice were anethesized and perfused through the heart with DMEM. The tumor and other organs were dissected, weighed. The tissues were homogenized in ice-cold DMEM containing protease inhibitor cocktail and 0.1% BSA. After centrifugation to remove the tissue debris bound phages were rescued by mixing the supernatant with 0.5 ml of ER2537 bacteria (New England Biolabs, USA) for 30 min at 37° C. The supernatant containing phage was diluted in Luria Broth (LB), after which aliquots were plated on LB agar plates containing X-Gal and IPTG. As control, unselected phage was administered at the same titer in the second group of animals.

Peptides

The poly-L-lysine-SIPVKFNKP-MG11 ((K₁₆)-MG11) and poly-L-lysine-SIPVKFNKP-H42 ((K₁₆)-H42) peptides were synthesized and purified (Mimotopes, Victoria, Australia). The peptides were dissolved in 0.15 M sodium chloride (NaCl) at 1 mg/ml and stored in small aliquots at −20° C. The purity of both peptides was >90%.

Formation of Peptide/DNA Complexes

The formation and transfection of peptide/DNA complexes were carried out according to Patel et al. with slight modifications. (Patel et al., 2001). Peptide/DNA complexes were prepared at a weight/weight ratio of 2:1 peptide: DNA. Plasmid DNA was diluted to 10 μg/ml in Ringer's buffer (B. Braun Melsungen AG, Melsungen, Germany) The appropriate volume of peptide was also diluted in Ringer's buffer to give a final concentration of 20 pg/ml (2:1 peptide: DNA ratio). The peptide was added dropwise to the DNA solution while vortexing gently (Stuart Scientific, UK). This mixture was allowed to incubate at room temperature (RT) for 30 min. After 30 min, the mixture was diluted to 4 μg/ml DNA with DMEM in the presence of 100 μM of chloroquine, and added to cells.

Transfection of Tumor Cell Lines

The human tumor cell lines, dGli36, SF767, U251, A549, HeLa, HepG2, and CNE2 were cultured in DMEM supplemented with 10% fetal bovine serum (FBS) (Hyclone Laboratories, Logan, Utah). For transfection, 3×10⁵ cells were seeded into each well of a six-well plate (Nunc, Roskilde, Denmark). After culturing the cells overnight, the cells were washed twice with PBS. Freshly prepared peptide/DNA complexes were added into each well and incubated for 2 h at 37° C. For transfection performed in the presence of serum, FBS was added to a final concentration of 10% directly after addition of peptide/DNA complexes to each well. The reporter gene activity was assayed after 24 h or 48 h.

Assay for Luciferase Activity

Cells were harvested from 6-well plates 24 h or 48 h following transfection, washed, resuspended in 120 μl of Tris-HCl (pH 7.8), and freeze-thawed three times. Cell debris was discarded following centrifugation at 14,000 g at 4° C. for 10 min. One hundred μl of the supernatant collected was used for assaying luciferase activity using the Auto-Lumat LB952 luminometer (EG&G Berthold, Bad Wildbad, Germany). Five microliter of the supernatant collected was used for the determination of protein concentration using the Bio-Rad protein assay dye reagent (Bio-Rad Laboratories, Hercules, USA) with the Ultrospec 3000 UV/visible spectrophotometer (Amersham Biosciences AB, Uppsala, Sweden).

In Vitro Fluorescent Peptide Binding Assay

For in vitro binding assay, 1×10⁵ cells were seeded into each well of a 24-well dish (Nunc, Roskilde, Denmark). After culturing the cells overnight at 37° C., the cells were washed once with PBS, followed by incubating in blocking buffer containing PBS with 1% BSA at room temperature for 1 h. The cells were washed twice with PBS. One hundred nanogram of lissamine-rhodamine conjugated peptide (Mimotopes, Victoria, Australia) were added to the blocking buffer containing 0.1% sodium azide, and incubated with the cells at 37° C. for 20 min. This is followed by 5 washes of PBS with 0.1% Tween-20 at RT for 5 min intervals. The cells were then fix in 4% paraformaldehyde, followed by counterstaining using FITC-phalloidin (Sigma-Aldrich Corp., St. Louis, Mo.) and mounted. Images were captured digitally and analyzed using LSM 510 confocal microscope (Carl Zeiss Microscopy, Gottingen, Germany) with appropriate filters.

In Vivo Fluorescent Peptide Binding Assay

SF767-tumor bearing mice were randomized into 2 groups (2 per group). Fifty microliter of 100 μg of lissamine rhodamine conjugated (K₁₆)-MG11 or (K₁₆)-H42 was injected intratumorally into 2 mice harboring the tumor, and allowed to circulate for 20 min. After 20 min, the mice were anethesized and perfused through the heart with PBS followed by 4% paraformaldehyde. The tumor were dissected and prepared for cryostat sections. The sections were then counterstained in FITC-phalloidin and mounted. Images were examined using LSM 510 meta confocal microscope (Carl Zeiss Microscopy, Göttingen, Germany) with appropriate filters.

Statistical Analysis

Data are presented throughout this study as means +standard error of the mean. Statistical significance was evaluated by paired t-test, and p<0.05 was considered significant.

### Results

Enrichment of “Glioma-Specific” Phage by In Vitro Biopanning

The present inventors employed biopanning to identify peptide sequences that bind specifically to human glioma cells. The Ph.D-12 phage display library, with a complexity of approximately 2×10⁹ sequences, was employed for screening peptides that would bind specifically to human glioma cells. This library consists of 12 random amino acid sequences (12-merpeptide) fused to the N-terminus of the minor coat protein of the M13 phage, in a valency of 5 copies per virion. The library (Ph.D-12) was screened against a mixture of human glioma cell lines including dGli36, SF767, U87MG, U251MG and U373MG at 37° C. to increase the probability of obtaining sequences that could interact generically with most glioma cells. Phages obtained were further subjected to negative panning using non-glioma cells lines, A549 (human lung carcinoma); CNE2 (human nasopharyngeal carcinoma); and HepG2 (human hepatoma) to eliminate non-specific background binding. The amino acid sequences of the 79 phage clones obtained following such a selection revealed several dominant sequence motifs (Table 1). The sequence SGHQLLLNKMPN, designated as MG2, was found in 24% of the clones; and LWATFPPRPPWL, designated as MG11, was found in 19% of the clones (Table 1). The BLAST search of MG2 demonstrated 60% sequence homology with the human mitogen-activated protein kinase kinase kinase 12 (MAP3K12). MAP3K12 was found to be highly expressed in brain and kidney, and has been implicated as an activator of the JNK/SAPK pathway (Su and Karin, 1996). Analysis of the second most frequently isolated phage-bearing peptide sequence, designated as MG11, showed a 64% homology to semaphorin 4B precursor protein. Semaphorin 4B is a member of the semaphorin super family, which is involved in the inhibition of axonal extension by providing local signals to specify territories inaccessible for growing axons (Rieger et al., 2003). In addition to phage-bearing the MG11 peptide sequence, the inventors have also isolated two other phage clones harboring peptide sequences that match to the semaphorin family of proteins. One of the sequence, HHGHSPTSPQVR, matches perfectly to the protein sequence of semaphorin 3A precursor protein, while the other peptide sequence, LPYGTSNRHAPV, showed a 64% homology to semaphorin 6B precursor protein. Semaphorin 3A, which binds with high affinity to neurophilin-1, induces the collapse and paralysis of neuronal growth cones, and binds to neuropilin-1 (Rieger et al., 2003). Neuropilin-1 is a receptor for the vascular endothelial growth factor family and has been implicated in blood vessel formation. It also functions as a co-receptor with the Flk-1/KDR receptor tyrosine kinase (Gluzman-Poltorak et al., 2000). The inventors noticed that several groups of the peptides selected are homologous to members of the semaphorin super family. It was previously reported that the semaphorin family of proteins play important roles during neural development (Schumacher et al., 2002; Rieger et al., 2003), since MG11 is the most common sequence identified among the group of phages isolated, MG11 was thus chosen for subsequent studies.

Characterization of the Binding Epitopes of MG11 Phage

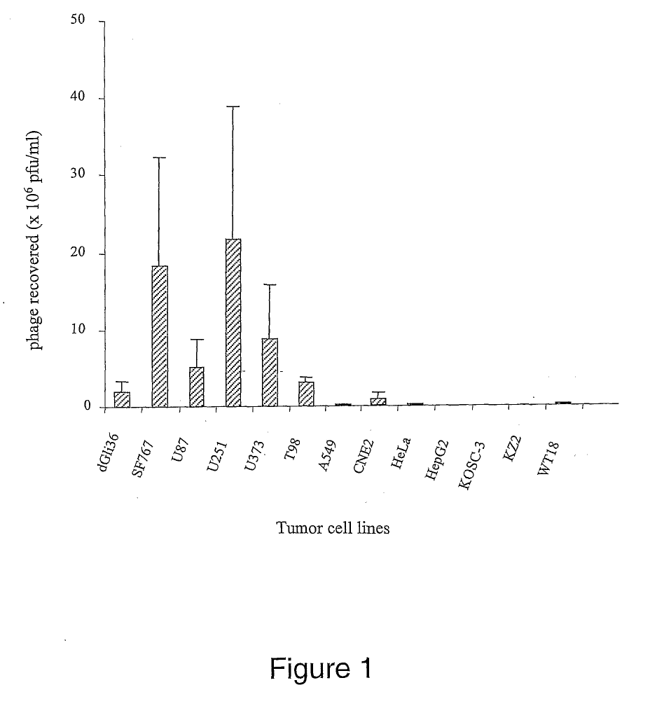

To determine if MG11 phage binds specifically to glioma cells, MG11 phage was incubated with tumor cells lines of various histotypes. These include the dGli36, SF767, U87MG, U251MG, U373MG and T98G glioma cells, and non glioma cells A549, CNE2, HeLa, HepG2, KOSC-3, KZ2 and WT18 as shown in FIG. 1. The efficiency of MG11 phage binding to glioma cells, ranges from 2×10⁶ plaque forming unit (pfu)/ml to 2.2×10⁷ pfu/ml, was at least 3-fold higher than non-glioma cells (FIG. 1). None of the non-glioma origins tumor cell lines tested exhibit considerable binding (ranges from 1.5×10⁴ pfu/ml to 9.6×10⁵ pfu/ml). Thus, in vitro phage binding assay indicated that MG11 phage binds specifically to glioma cells.

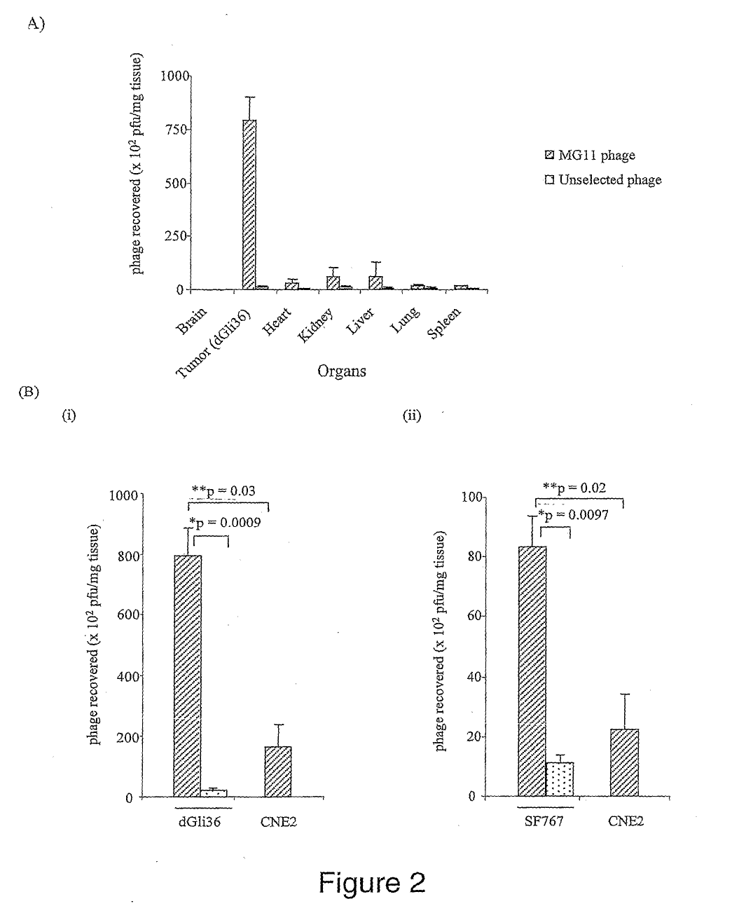

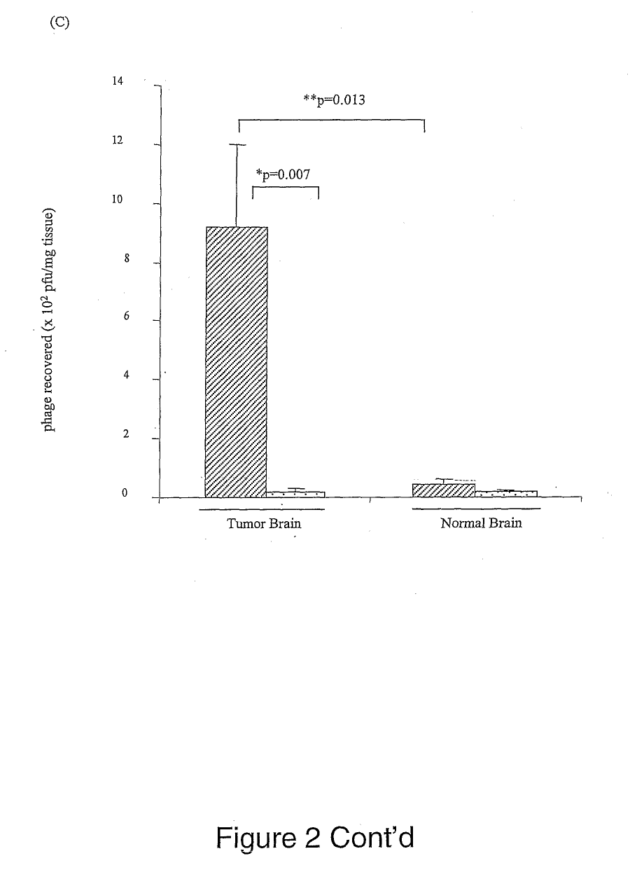

To determine if phage bearing the MG11 binding motif can target specifically in vivo to human glioma xenograft, 10¹² pfu of MG11 phage was injected via tail-vein to immunodeficient SCID mice harboring s.c. dGli36-derived glioma xenograft. Recovery of phage particles from tissues as well as xenografted dGli36 tumor indicated that MG11 phage is specific for tumor only (7.94×10⁴ pfu/mg), whereas other tissues including brain, heart, kidney, liver, lung and spleen gave only background binding (FIG. 2A). Unselected phage was employed as control and minimal to no binding could be detected (FIG. 2A). The tumor specificity of MG11 was also examined by administering MG11-phage via tail vein to immunodeficient mice bearing s.c. glioma xenograft (dGli36) and non-glioma xenograft (CNE2) on the right and left hind thigh, respectively. The enrichment factor in homing to the s.c. dGli36 glioma xenograft (7.9×10⁴ pfu/mg) as compared to the s.c. CNE2 (1.67×10⁴ pfu/mg) tumor is 5-fold, p=0.03 (FIG. 2B(i)). Similar results were obtained when another human glioma SF767 was employed. The enrichment factor of MG11 phage to SF767-derived tumor (8.34×10³ pfu/mg) is 4-fold higher when compared with the CNE2-derived tumor (2.21×10³ pfu/mg), p=0.02 (FIG. 2B (ii)), indicating that the binding of MG11 to human gliomas cell lines is specific.

The specificity of MG11 phage binding to human glioma cells in the context of normal mouse astrocytes was investigated. 10¹² phage were injected via tail vein into immunodeficient nude mice bearing an intracranial xenograft of dGli36 human glioma cells in the right hemisphere. Phage recovered from the right hemisphere bearing the xenografted tumor (9.2×10² pfu/mg) in mice injected with the MG11 phage gave an enrichment of 20-fold compared to those recovered from the normal side of the brain (0.5×10² pfu/mg) (left hemisphere) (p=0.013) (FIG. 2C). No significant difference could be detected in the control groups that were injected with unselected phage (FIG. 2C). These results demonstrated that phage bearing the MG11 epitope target specifically to cells of glial origins and do not bind to the normal brain cells.

In Vitro Specificity of (K₁₆)-MG11 in Human Tumor Cell Lines

The amino acid residues that make up the MG11 peptide sequence are very hydrophobic and could not be easily purified. To facilitate the purification procedure, we have inserted 16 lysine residues to the sequence. The addition of poly-lysine residues also enhance the potential of the peptide to interact with the negatively charged plasmid DNA as demonstrated by Patel et al (Patel et al., 2001). In addition to the poly-lysine residues, a spacer sequence identical to that used by Patel et al. were also included to facilitate the movement of the peptide.

The peptide, (K₁₆)-MG11, comprising a chain of sixteen consecutive lysine residue, to allow electrostatic interaction with DNA, synthesized along with the MG11 peptide sequence, LWATFPPRPPWL, was chemically synthesized and complexed with a luciferase reporter plasmid, PNGVL-Luciferase (pNGVL-Luc). To determine the optimal concentration that would allow DNA and peptide interactions and to maximize the efficiency of gene uptake, various peptides to DNA ratio were studied and employed to transfect SF767 cells.

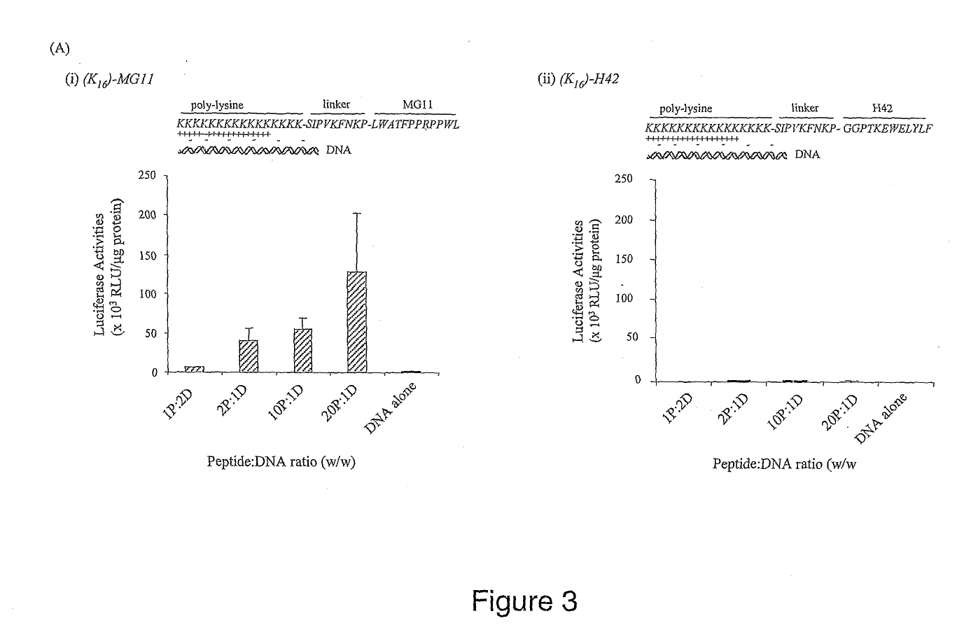

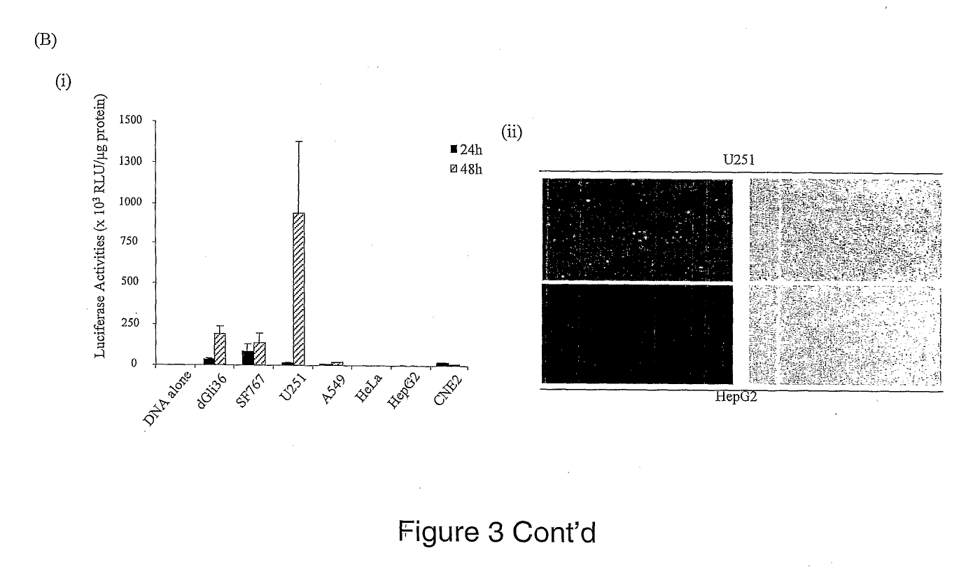

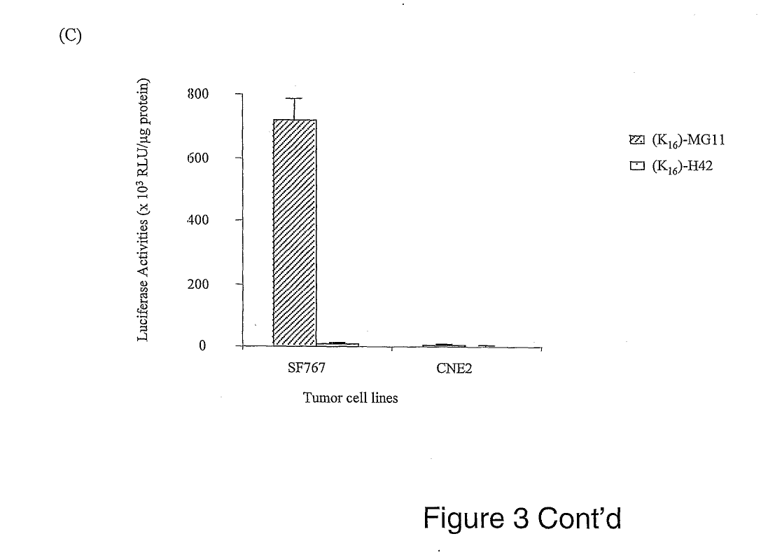

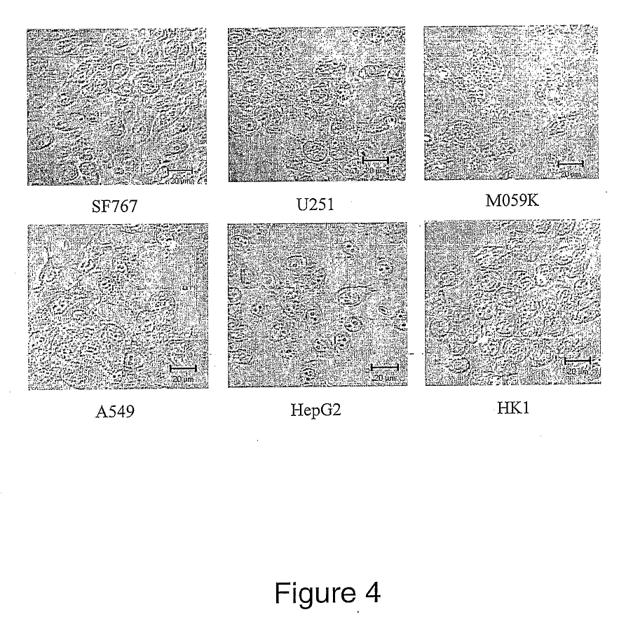

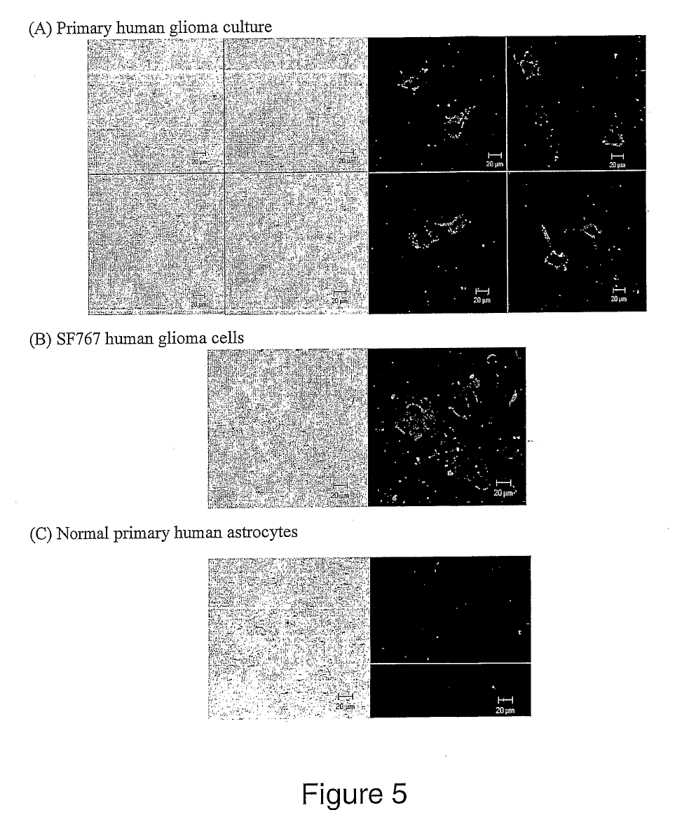

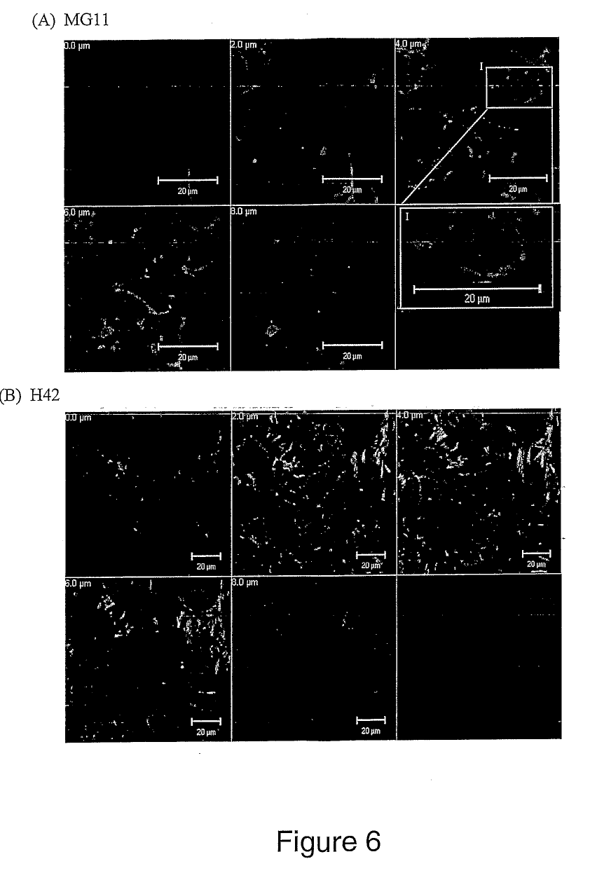

Varying ratios of the pNGVL-Luc plasmid and (K₁₆)-MG11 peptide was complexed followed by incubation in SF767 glioma cells in the presence of chloroquine. Chloroquine, was added as it has been previously demonstrated to prevent lysosomal DNA degradation (Zauner et al., 1997). After 24 h, luciferase expression was detected (39803 RLU/μg protein) using peptide: DNA ratio (weight: weight) of 2:1 (FIG. 3A (i)). The level of luciferase expression increases with escalating ratios of peptide to DNA (FIG. 3A (i)). This suggests that perhaps the abundance of lysine residues would facilitate the interaction of the peptide with the DNA molecules and hence ensure that majority of the DNA molecules is complexed with the peptide. At peptide: DNA ratios of 10:1 and 20:1, toxicity could be detected and the treated SF767 glioma cells were not as healthy as that of the controls. This could be due to the presence of high concentration of polylysine, which is known to have toxic effect on cells (Zauner et al., 1997). For subsequent experiments, we have therefore adopted the optimal peptide: DNA ratio of 2:1.

To rule out the possibility that the binding of the peptide to the cells is due to the lysine residues inserted, the inventors synthesized a control peptide in their experiment to confirm that the luciferase activities observed were a consequence of targeting. A control peptide, comprising of the 16 lysine residue followed by 12 amino acid sequence derived from biopanning of a non-glioma cell line, (K₁₆)-H42, was also synthesized. H42 bears the sequence GGPTKEWELYLF. Similar experiments were carried out using the luciferase DNA and (K₁₆)-H42 at various peptides to DNA ratios and no significant binding could be detected when analyzed 24 h post-transfection (FIG. 3A (ii)).

To further demonstrate the specificity of (K₁₆)-MG11 peptide on glioma cells and to determine the optimal time point of luciferase transgene expression, peptide/DNA complex, prepared from mixing (K₁₆)-MG11 peptide to pNGVL-Luc DNA at 2:1 (w/w) ratio, were added to human tumor cell lines of different histotypes. These include dGli36, SF767, U251MG, U373MG, A549, HeLa, HepG2 and CNE2 cells. Binding of peptide/DNA complexes was performed in the presence of 100 μM of chloroquine, and luciferase expression was assayed 24 h and 48 h following transfection. It was observed that 24 h post-transfection, the luciferase activity was at least 4-fold higher in SF767 glioma cell line (85473 RLU/μg) when compared with CNE2 cells (19849 RLU/μg) (FIG. 3B (i)). It was also determined that the level of gene expression at 48 h post-delivery is much higher than that of the 24 h (FIG. 3B (i)). The level of luciferase expression detected in U251 glioma cell line increased dramatically in 48 h following transfection (FIG. 3B (i)). Similar results were observed with the human glioma cells dGli36 and SF767 (FIG. 3B (i)). Similar experiments using plasmid DNA bearing the reporter gene enhanced green fluorescent protein (eGFP) and the (K₁₆)-MG11 peptide also gave similar results (FIG. 3B (ii)). Strong green fluorescence could be detected in U251 glioma cells but not in HepG2 human hepatocellular carcinoma cells.

Furthermore, when peptide/DNA complexes prepared from (K₁₆)-MG11 or (K₁₆)-H42 with pNGVL-Luc was mixed and added to SF767 and CNE2 cells in the presence of chloroquine, the luciferase expression in SF767 glioma cells transfected with (K₁₆)-MG11: pNGVL-Luc (716940 RLU/pg) is 84-fold higher than that of (K₁₆)-H42: pNGVL-Luc (8444 RLU/μg) control peptide transfected cells, demonstrating that the binding of (K₁₆)-MG11 peptide is specific, and not due to the presence of positive charge lysine residues (FIG. 3C). When compared to non-glioma cell line, CNE2, the luciferase expression in (K₁₆)-MG11 transfected glioma cells is 151-fold higher in SF767 cells than CNE2 cells (FIG. 3C). These results clearly illustrate that (K₁₆)-MG11 is specific for glioma cells.

Characterization of (K₁₆)-MG11 Peptide Targeted Delivery In Vitro and In Vivo

To determine the direct specific binding of the (K₁₆)-MG11 peptide to cell surface of glioma cells, (K₁₆)-MG11 peptide was conjugated to lissamine rhodamine. After incubating the conjugated (K₁₆)-MG11 to the various tumor cell lines, SF767, U251MG, M059K, A549, HepG2, and HK1, for 20 minutes at 37° C., the cells were examined under confocal microscopy and the level of fluorescence detected were compared between glioma and non-glioma cells. SF767, U251MG, and M059K human glioma cell lines exhibited detectable fluorescence as shown by the presence of rhodamine on the surface of the cells (FIG. 4 upper panel). Little or no fluorescence was observed with non-glioma cell lines (FIG. 4 lower panel). In contrast, non-specific red fluorescence was observed in all cell lines studied when incubated with (K₁₆)-H42 control peptide under similar conditions (data not shown). Unlike the (K₁₆)-MG11 peptide, no discrimination between glioma and non-glioma cells could be ascertained with the rhodamine-conjugated (K₁₆)-H42 peptide (data not shown).

The binding specificity of (K₁₆)-MG11 peptide to primary human glioma cells and normal primary human astrocytes was determined subsequently. Primary human glioma cells derived from biopsy or normal primary human astrocytes were incubated with lissamine-rhodamine conjugated (K₁₆)-MG11 peptide at 37° C. for 20 min. Significant binding of the (K₁₆)-MG11 peptide was observed only in primary human glioma cells (FIG. 5A). This binding specificity is comparable to that observed in SF767 human glioma cell line (FIG. 5B). In contrast, no fluorescence could be detected with the normal primary human astrocytes under similar conditions (FIG. 5C). These results clearly demonstrated that the MG11 peptide is specific for human glioma cells and do not bind to normal human astrocytes.

To further demonstrate the tumor specificity of the (K₁₆)-MG11 peptide on glioma xenograft, SCID mice harboring s.c.-established SF767-derived glioma xenograft were injected intratumorally with lissamine-rhodamine conjugated (K₁₆)-MG11 peptide and (K₁₆)-H42 peptide. After 20 min of circulation, the mouse bearing the SF767 glioma xenograft was perfused and tumor was harvested. The tumor sections were counterstained with FITC-labeled phalloidin, which stains F-actin, to determine the cell surface. Cryostat sections of the tumors showed red fluorescence binding in SF767 glioma xenograft injected with (K₁₆)-MG11 peptide (FIG. 6A), demonstrating the binding of the (K₁₆)-MG11 peptide. This binding as observed in the insert (I) indicated that the (K₁₆)-MG11 peptide interacted with cell surface receptor on the tumor cells. In contrast, red fluorescent was not detected in the SF767 tumor injected with the (K₁₆)-H42 peptide (FIG. 6B). Taken together, these results suggested that the observed fluorescence can be attributable to the specific binding and subsequent uptake of the (K₁₆)-MG11 peptide.

Discussion

The inventors report herein the isolation and characterization of novel glioma-specific peptides that can target specifically to a wide array of human glioma cells in vitro and in vivo. They have employed the phage display peptide library to isolate phages bearing peptide sequences that bind specifically to human glioma cell lines. Analysis of the 79 phage clones obtained following biopanning of the Ph.D. 12 phage library revealed several dominant sequence motifs (Table 1). The sequence SGHQLLLNKMPN encoded by the phage MG2 was found in 24% of the clones and this sequence was shown to have 60% homology to the human mitogen activated protein kinase kinase kinase 12 (MAP3K12). The second most frequently isolated phage clone, MG11, bears the sequence of LWATFPPRPPWL, which matches to the semaphorin 4B precursor, a membrane-bound semaphorin. Plexins, which form complexes with neuropilin, appear to be the receptors for this type of semaphorins (Tamagnone et al., 1999). Interestingly, two other phage clones that harbor peptide sequences which shown homology to the semaphorin family of proteins were also isolated. The sequence HHGHSPTSPQVR and LPYGTSNRHAPV matches to the protein sequence of the secreted semaphorin 3A precursor protein and the membrane-bound semaphorin 6B precursor protein, respectively. Semaphorin 3A induces the collapse and paralysis of neuronal growth cones, and binds with high affinity to neuropilin-1 (Rieger et al., 2003), a receptor for the vascular endothelial growth factor family (Miao et al., 2000). It was previously reported that glioma cell lines not only expresses neuropilins, but also plexins. Together, these two molecules form complexes with the class 3 semaphorins and are necessary for the morphological changes induced (Tamagnone et al., 1999). Semaphorins not only modulate the extension of the axonal cones but also the migration of neural progenitor cells (Marin et al., 2001). As glioma cells are characterized by its ability to infiltrate and migrate to distance sites within the brain, it is speculated that semaphorins might play a role in cancer development. Furthermore, RT-PCR analysis of glioma cell lines demonstrated the presence of semaphorin 3 and semaphorin 6B in human glioma cell lines (Correa et al., 2001; Rieger et al., 2003), consistent with reports of the presence of semaphorins in tumor progression of mouse mammary and human metastatic lung adenocarcinoma (Martin-Satue and Blanco, 1999). The semaphorin family of proteins are known to play important roles during neural development (Tamagnone and Comoglio, 2000), since MG11 is the most common sequence identified (Table 1), MG11 was chosen for subsequent studies. Sequences that target to human malignant glioma cells have been demonstrated for glioma cell lines (Spear et al., 2001; Zhang et al., 2001). However, their specificity is limited and confined to a single cell line. Spear et. al. reported a peptide that is specific for U87MG glioma cell line and having the sequence, MCPKHPLGC, but did not bind to other glioma cells (Spear et al., 2001). Zhang et. al. reported a sequence that binds to glioma cells. However, this sequence is not specific for gliomas and it also binds to other cancer cell lines (Zhang et al., 2001). In the present case, phage bearing the MG11 sequence gave an in vitro enrichment binding of over 5-fold for glioma cells in comparison to non-glioma cells (FIG. 1). Consistent with the in vitro data, the peptide-encoding phage MG11 is able to target specifically to glioma xenograft, with an enrichment factor of five over non-glioma tumor xenograft (FIG. 2B). The specificity of MG11 to bind to tumor of glial origins was further supported in vitro using MG11 peptide sequence to direct luciferase reporter gene expression in glioma cells but not in non-glioma cells (FIG. 3). Furthermore, lissamine-rhodamine conjugated MG11 peptide injected intratumorally is specifically targeted to glioma xenograft, but not against xenografts of non-glioma-derived cancer (FIG. 6).

One of the challenges for in vivo targeting is to be able to direct the binding of the targeting peptide to tumor cells instead of normal cells. The inventors have shown that the MG11 peptide only binds to human glioma-derived cell lines (FIG. 4) and primary human glioma cells (FIG. 5A) but not to normal human brain cells (FIG. 5C) or other non-glioma cells (FIG. 4). These observations would be of great relevance in designing strategies for targeted therapy of glioma.

The isolation of the MG11 peptide provides the possibilities to conjugate therapeutic DNA or drugs directly to the peptides for targeting. This could lower the therapeutic dose of either DNA or drugs and thus reduce any potential harmful side-effects due to high level of therapeutic DNA or drugs. However, to effectively deliver therapeutic DNA into the cells, the uptake, intracellular trafficking and nuclear retention of plasmid DNA must be achieved. Cationic lipids, viral vector or polymers are some of the strategies that could be employed to coupled to targeting molecules, to increase their stability (Watkins, 1997; Mahat et al., 1999; Nicklin, 2000; Patel et al., 2001). Addition of chloroquine to the cells could prevent the targeting peptide/DNA complexes from lysosomal degradation, thus enhancing the effectiveness of the gene delivery system (Zauner et al., 1997).

### REFERENCES

AGHA-MOHAMMADI, S., and LOTZE, M. T. (2000). Regulatable systems: applications in gene therapy and replicating viruses. J Clin Invest 105, 1177-1183.

ARAP, W., HAEDICKE, W., BERNASCONI, M., KAIN, R., RAJOTTE, D., KRAJEWSKI, S., ELLERBY, H. M., BREDESEN, D. E., PASQUALINI, R., and RUOSLAHTI, E. (2002a). Targeting the prostate for destruction through a vascular address. Proc Natl Acad Sci U S A 99, 1527-1531.

ARAP, W., KOLONIN, M. G., TREPEL, M., LAHDENRANTA, J., CARDOVILA, M., GIORDANO, R. J., MINTZ, P. J., ARDELT, P. U., YAO, V. J., VIDAL, C. I., CIIEN, L., FLAMM, A., VALTANEN, H., WEAVIND, L. M., HICKS, M. E., POLLOCK, R. E., BOTZ, G. H., BUCANA, C. D., KOIVUNEN, E., CAHILL, D., TRONCOSO, P., BAGGERLY, K. A., PENTZ, R. D., DO, K. A., LOGOTHETIS, C. J., and PASQUALINI, R. (2002b). Steps toward mapping the human vasculature by phage display. Nat Med 8, 121-127.

ARAP, W., PASQUALINI, R., and RUOSLAHTI, E. (1998). Cancer treatment by targeted drug delivery to tumor vasculature in a mouse model. Science 279, 377-380.

BANSAL, K., and ENGELHARD, H. (2000). Gene therapy for brain tumors. Current Oncology Reports 2, 463-472.

BARRY, M. A., DOWER, W. J., and JOHNSTON, S. A. (1996). Toward cell-targeting gene therapy vectors: selection of cell-binding peptides from random peptide-presenting phage libraries. Nat Med 2, 299-305.

CAMPA, M. J., SERLIN, S. B., and PATZ, E. F., JR. (2002) Development of novel tumor imaging agents with phage-display combinatorial peptide libraries. Acad Radiol 9, 927-932.

COHEN, K., LIU, T., BISSONETTE, R., PURI, R., and FRANKEL, A. (2003). DAB389EGF fusion protein therapy of refractory glioblastoma multiforme. Curr Pharm Biotechnol 4, 39-49.

CORREA, R., SASAHARA, R., BENGTSON, M., KATAYAMA, M., SALIM, A., BRENTANI, M., SOGAYAR, M., DE SOUZA, S., and SIMPSON, A. (2001). Human semaphorin 6B [(HSA)SEMA6B], a novel human class 6 semaphorin gene: alternative splicing and all-trans-retinoic acid-dependent downregulation in glioblastoma cell lines. Genomics 73, 343-348.

COWAN, W., HARTER, D., and KANDEL, E. (2000). The emergence of modern neuroscience: some implications for neurology and psychiatry. Annual Review in Neuroscience 23, 343-391.

ESSLER, M., and RUOSLAHTI, E. (2002). Molecular specialization of breast vasculature: a breast-homing phage-displayed peptide binds to aminopeptidase P in breast vasculature. Proc Natl Acad Sci U S A 99, 2252-2257.

FREDERIKSEN, K. S., ABRAHAMSEN, N., CRISTIANO, R. J., DAMSTRUP, L., and POULSEN, H. S. (2000). Gene delivery by an epidermal growth factor/DNA polyplex to small cell lung cancer cell lines expressing low levels of epidermal growth factor receptor. Cancer Gene Ther 7, 262-268.

GLUZMAN-POLTORAK, Z., COHEN, T., HERZOG, Y., and NEUFELD, G. (2000). Neuropilin-2 and Neuropilin-1 are receptors for the 165-amino acid form of vascular endothelial growth factor (VEGF) and of placenta growth factor-2, but only neuropilin-2 functions as a receptor for the 145-amino acid form of VEGF. J. Biol. Chem. 275, 18040-18045.

KOIVUNEN, E., ARAP, W., RAJOTTE, D., LAHDENRANTA, J., and PASQUALINI, R. (1999). Identification of receptor ligands with phage display peptide libraries. J Nucl Med 40, 883-888.

LAQUERRE, S., ANDERSON, D. B., STOLZ, D. B., and GLORIOSO, J. C. (1998). Recombinant herpes simplex virus type 1 engineered for targeted binding to erythropoietin receptor-bearing cells. J Virol 72, 9683-9697.

LIU, R., ENSTROM, A. M., and LAM, K. S. (2003). Combinatorial peptide library methods for immunobiology research. Exp Hematol 31, 11-30.

MAHAT, R., MONERA, O., SMITH, L., and ROLLAND, A. (1999). Peptide-based gene delivery. Curr Opin Mol Ther 1, 226-243.

MARIN, O., YARON, A., BAGRI, A., TESSIER-LAVIGNE, M., and RUBENSTEIN, J. L. (2001). Sorting of striatal and cortical interneurons regulated by semaphorin-neuropilin interactions. Science 293, 872-875.

MARTIN-SATUE, M., and BLANCO, J. (1999). Identification of semaphorin E gene expression in metastatic human lung adenocarcinoma cells by mRNA differential display. J Surg Oncol 72, 18-23.

MIAO, H. Q., LEE, P., LIN, H., SOKER, S., and KLAGSBRUN, M. (2000). Neuropilin-1 expression by tumor cells promotes tumor angiogenesis and progression. Faseb J 14, 2532-2539.

MISCHEL, P., and CLOUGHESY, T. (2003). Targeted molecular therapy of GBM. Brain Pathology 13, 52-61.

NETTELBECK, D. M., JEROME, V., and MULLER, R. (2000). Gene therapy: designer promoters for tumour targeting. Trends Genet 16, 174-181.

NICKLIN, S., WHITE, S., WATKINS, S., HAWKINS, R., AND BAKER, A. (2000). Selective targeting of gene transfer to vascular endothelial cells by use of peptides isolated by phage display. Circulation 102, 231-137.

OHNO, K., SAWAI, K., IIJIMA, Y., LEVIN, B., and MERUELO, D. (1997). Cell-specific targeting of Sindbis virus vectors displaying IgG-binding domains of protein A. Nat Biotechnol 15, 763-767.

PARMLEY, S., and SMITH, G. (1989). Antibody-selectable filamentous fd phage vectors: affinity purification of target genes. Gene 73, 305-318.

PASQUALINI, R., KOIVUNEN, E., KAIN, R., LAHDENRANTA, J., SAKAMOTO, M., STRYHN, A., ASHMUN, R. A., SHAPIRO, L. H., ARAP, W., and RUOSLAHTI, E. (2000). Aminopeptidase N is a receptor for tumor-homing peptides and a target for inhibiting angiogenesis. Cancer Res 60, 722-727.

PASQUALINI, R., and RUOSLAHTI, E. (1996). Organ targeting in vivo using phage display peptide libraries. Nature 380, 364-366.

PATEL, S., ZHANG, X., COLLINS, L., and FABRE, J. W. (2001). A small, synthetic peptide for gene delivery via the serpin-enzyme complex receptor. J Gene Med 3, 271-279.

RAINOV, N., and KRAMM, C. (2001). Vector delivery methods and targeting strategies for gene therapy of brain tumors. Current Gene Therapy 1, 367-383.

RAINOV, N., and REN, H. (2003). Gene therapy for human malignant brain tumors. Cancer Journal 9, 180-188.

RAJOTTE, D., ARAP, W., HAGEDORN, M., KOIVUNEN, E., PASQUALINI, R., and RUOSLAHTI, E. (1998). Molecular heterogeneity of the vascular endothelium revealed by in vivo phage display. J Clin Invest 102, 430-437.

RASMUSSEN, U., SCHREIBER, V., SCHULTZ, H., MISCHLER, F., and SCHUGHART, K. (2002). Tumor cell-targeting by phage-displayed peptides. Cancer Gene Ther 9, 606-612.

REDDY, J. A., ABBURI, C., HOFLAND, H., HOWARD, S. J., VLAHOV, I., WILS, P., and LEAMON, C. P. (2002). Folate-targeted, cationic liposome-mediated gene transfer into disseminated peritoneal tumors. Gene Ther 9, 1542-1550.

RIEGER, J., WICK, W., and WELLER, M. (2003). Human Malignant Glioma Cells Express Semaphorins and Their Receptors, Neuropilins and Plexins. Glia 42, 379-389.

SAMOYLOVA, T., MORRISON, N., and COX, N. (2003). Molecular markers of glial tumors: current targeting strategies. Curr Med Chem 10, 831-843.

SCHUMACHER, T., HOFER, S., EICHHORN, K., WASNER, M., ZIMMERER, S., FREITAG, P., PROBST, A., GRATZL, O., REUBI, J.-C., MAECKE, H., MUELLER-BRAND, J., and MERLO, A. (2002). Local injection of the 90Y-labelled peptidic vector DOTATOC to control gliomas of WHO grades II and III: an extended pilot study. European Journal of Nuclear Medicine 29, 486-493.

SHIBATA, T., GIACCIA, A., and BROWN, J. (2000). Development of a hypoxia-responsive vector for tumor-specific gene therapy. Gene Therapy 7, 493-498.

SPEAR, M., BREAKEFIELD, X., BELTZER, J., SCHUBACK, D., WEISSLEDER, R., PARD, F., and LADNER, R. (2001). Isolation, characterization, and recovery of small peptide phage display epitopes selected against viable malignant glioma cells. Cancer Gene Ther 8, 506-511.

SU, B., and KARIN, M. (1996). Mitogen-activated protein kinase cascades and regulation of gene expression. Current Opinion in Immunology 8, 402-411.

SZARDENINGS, M., TORNROTH, S., MUTULIS, F., MUCENIECE, R., KEINANEN, K., KUUSINEN, A., and WIKBERG, J.E. (1997). Phage display selection on whole cells yields a peptide specific for melanocortin receptor 1. J Biol Chem 272, 27943-27948.

TAI, C. K., LOGG, C. R., PARK, J. M., ANDERSON, W. F., PRESS, M. F., and KASAHARA, N. (2003). Antibody-mediated targeting of replication-competent retroviral vectors. Hum Gene Ther 14, 789-802.

TAMAGNONE, L., ARTIGIANI, S., CHEN, H., HE, Z., MING, G. I., SONG, H., CHEDOTAL, A., WINBERG, M. L., GOODMAN, C. S., POO, M., TESSIER-LAVIGNE, M., and COMOGLIO, P. M. (1999) Plexins are a large family of receptors for transmembrane, secreted, and GPI-anchored semaphorins in vertebrates. Cell 99, 71-80.

TAMAGNONE, L., and COMOGLIO, P. M. (2000). Signaling by semaphorin receptors: cell guidance and beyond. Trends Cell Biol 10, 377-383.

TREPEL, M., ARAP, W., and PASQUALINI, R. (2002). In vivo phage display and vascular heterogeneity: implications for targeted medicine. Curr Opin Chem Biol 6, 399-404.

UENO, M., KOYAMA, F., YAMADA, Y., FUJIMOTO, H., TAKAYAMA, T., KAMADA, K., NAITO, A., HIRAO, S., MUKOGAWA, T., HAMADA, H., and NAKAJIMA, Y. (2001). Tumor-specific chemo-radio-gene therapy for colorectal cancer cells using adenovirus vector expressing the cytosine deaminase gene. Anticancer Res 21, 2601-2608.

VAN BEUSECHEM, V. W., GRILL, J., MASTENBROEK, D. C., WICKHAM, T. J., ROELVINK, P. W., HAISMA, H. J., LAMFERS, M. L., DIRVEN, C. M., PINEDO, H. M., and GERRITSEN, W. R. (2002). Efficient and selective gene transfer into primary human brain tumors by using single-chain antibody-targeted adenoviral vectors with native tropism abolished. J Virol 76, 2753-2762.

WATKINS, S., MESYANZHINOV, V., KUROCHKINA, L., AND HAWKINS, R. (1997). The ‘adenobody’ approach to viral targeting: specific and enhanced adenoviral gene delivery. Gene Ther 10, 1004-1012.

WEYERBROCK, A., and OLDFIELD, E. (1999). Gene transfer technologies for malignant gliomas. Current Opinion in Oncology 11, 168-173.

WHITE, S. J., NICKLIN, S. A., SAWAMURA, T., and BAKER, A. H. (2001). Identification of peptides that target the endothelial cell-specific LOX-1 receptor. Hypertension 37, 449-455.

WU, G. Y., and WU, C. H. (1987). Receptor-mediated in vitro gene transformation by a soluble DNA carrier system. J Biol Chem 262, 4429-4432.

ZAUNER, W., KICHLER, A., SCHMIDT, W., MECHTLER, K., and WAGNER, E. (1997). Glycerol and polylysine synergize in their ability to rupture vesicular membranes: a mechanism for increased transferrin-polylysine-mediated gene transfer. Exp Cell Res 232, 137-145.

ZHANG, J., SPRING, H., and SCHWAB, M. (2001). Neuroblastoma tumor cell-binding peptides identified through random peptide phage display. Cancer Letters 171, 153-164.

TABLE 1

Lists of peptides isolated from biopanning of human glioma
cell lines.

Frequency of
occurrence
(% of total

| | | |
|---|---|---|
| clones | Homology to known | % |

| | | | | |
|---|---|---|---|---|
| Sequences | sequenced) | human proteins | Alignment | homology |

| | | | | | | | | |
|---|---|---|---|---|---|---|---|---|
| 1 | SGHQLLLNKMPN | 24% | Mitogen-activated | | | * *** ** ^{c} | | 60 |
| | (MG2) | | protein kinase | Db^{a} | 599 | HDLLLRKMSS | 608 | |
| | | | kinase kinase 12 | Qy^{b} | 3 | HQLLLNKMPN | 12 | |
| 2 | LWATFPPRPPWL | 19% | Semaphorin 4B | | | * ***** * | | 64 |
| | (MG11) | | precursor | D^{b} | 11 | WGALPPRPPLL | 21 | |
| | | | | Qy | 2 | WATFPPRPPWL | 12 | |
| 3 | WSAAPTKPPYHT | 6% | Voltage-dependent | | | * * *** | | 46 |
| | | | L-type calcium | Db | 1981 | WATPPATPPYR | 1991 | |
| | | | channel alpha-1D | Qy | 1 | WSAAPTKPPYH | 11 | |
| | | | subunit | | | | | |
| 4 | ILANDLTAPGPR | 5% | None | | | | | |
| 5 | HHGHSPTSPQVR | 4% | Semaphorin 3A | | | ****** | | 100 |
| | | | precursor | Db | 575 | HHGHSP | 580 | |
| | | | | Qy | 1 | HHGHSP | 6 | |
| 6 | LPYGTSNRHAPV | 4% | Semaphorin 6B | | | **** * ** | | 64 |
| | | | precursor | Db | 435 | LPYGGADRTAP | 444 | |
| | | | (Sema Z) | Qy | 1 | LPYGTSNRHAP | 11 | |
| 7 | YVQGWNYHDLTR | 4% | None | | | | | |
| 8 | LWAAFPPQASVA | 4% | Synaptojanin 1 | | | ****** * | | 70 |
| | | | | Db | 1245 | AAFPPQSSLP | 1254 | |
| | | | | Qy | 3 | AAFPPQASVA | 12 | |
| 9 | FDTPHTLTWPHG | 3% | None | | | | | |

^{a}Db represents sequence from database.
^{b}Qy is the query sequence from phage.
^{c}“*” represents identical amino acid.

1. A peptide having up to 30 amino acids in length and comprising a sequence selected from the group consisting of

| | |
|---|---|
| SGHQLLLNKMPN, | (SEQ ID NO. 1) |
| LWATFPPRPPWL, | (SEQ ID NO. 2) |
| WSAAPTKPPYHT, | (SEQ ID NO. 3) |
| and | |
| ILANDLTAPGPR, | (SEQ ID NO. 4) |
| HHGHSPTSPQVR, | (SEQ ID NO. 5) |
| LPYGTSWRHAPV, | (SEQ ID NO. 6) |
| YVQGWNYHDLTR, | (SEQ ID NO. 7) |
| LWAAFPPQASVA. | (SEQ ID NO. 8) |
| [[and]] | |
| FDTPHTLTWFHG. | (SEQ ID NO. 9) |

2. A peptide, the amino acid sequence of which consists of an amino acid sequence selected from the group consisting of:

| | |
|---|---|
| SGHQLLLNKMPN, | (SEQ ID NO. 1) |
| LWATFPPRPPWL, | (SEQ ID NO. 2) |
| WSAAPTKPPYHT, | (SEQ ID NO. 3) |
| and | |
| ILANDLTAPGPR, | (SEQ ID NO. 4) |
| HHGHSPTSPQVR, | (SEQ ID NO. 5) |
| LPYGTSWRHAPV, | (SEQ ID NO. 6) |
| YVQGWNYHDLTR, | (SEQ ID NO. 7) |
| LWAAFPPQASVA. | (SEQ ID NO. 8) |
| [[and]] | |
| FDTPHTLTWFHG | (SEQ ID NO. 9) |

3. A peptide having up to 30 amino acids in length comprising an amino acid sequence having at least 8 residues identical with corresponding residues in an amino acid sequence selected from the group consisting of:

| | |
|---|---|
| SGHQLLLNKMPN, | (SEQ ID NO. 1) |
| LWATFPPRPPWL, | (SEQ ID NO. 2) |
| WSAAPTKPPYHT, | (SEQ ID NO. 3) |
| and | |
| ILANDLTAPGPR, | (SEQ ID NO. 4) |
| HHGHSPTSPQVR, | (SEQ ID NO. 5) |
| LPYGTSWRHAPV, | (SEQ ID NO. 6) |
| YVQGWNYHDLTR, | (SEQ ID NO. 7) |
| LWAAFPPQASVA. | (SEQ ID NO. 8) |
| [[and]] | |
| FDTPHTLTWFHG | (SEQ ID NO. 9) |

wherein the peptide is capable of binding glioma cells.

4. A complex comprising a peptide according claim 3 associated with a therapeutic or diagnostic agent.

5. A complex according to claim 4 wherein the diagnostic agent is a protein.

6. A complex according to claim 4 wherein the diagnostic agent is a label.

7. A complex according to claim 6 wherein said label is selected from the group consisting of a radioactive label, a fluorescent label, chemiluminescent label and an enzyme label.

8. A complex according to claim 4 wherein the diagnostic agent is a nucleic acid sequence.

9. A complex according to claim 8 wherein the nucleic acid sequence encodes a label.

10. A complex according to claim 9 wherein the label is selected from the group consisting of radioactive label, a fluorescent label, chemiluminescent label and an enzyme label.

11. A complex according to claim 4 wherein the therapeutic agent is a cytotoxic agent.

12. A complex according to claim 4 wherein the therapeutic agent is a nucleic acid sequence capable of encoding a cytotoxic agent.

13. A complex according to claim 11 wherein the cytotoxic agent is selected from the group consisting of 6-Diazo-5-oxo-1/-norleucine [DON], Actinomycin D, Mitomycin, Mitoxantrone, Cisplatin, Melphalan, Etoposid, 5FU, antracyclines, Paclitaxel and Tamoxifen.

14. A complex according to claim 4 wherein the therapeutic agent is a nucleic acid sequence capable of encoding an enzyme for use in a prodrug activation systems.

15. A complex according to claim 14 wherein the prodrug activation system is viral thymidine kinase or Gancyclovir.

16. A complex according to claim 4 wherein the therapeutic agent is a chemokine or a nucleic acid sequence capable of encoding said chemokine.

17. A complex according to claim 4 wherein the therapeutic agent is an immuno-enhancer or a nucleic acid sequence capable of encoding said immuno-enhancer.

18. A complex according to claim 17 wherein the immuno- enhancer is melatonin.

19. A complex according to claim 4 which is a viral particle.

20. A pharmaceutical composition comprising a peptide according to claim 3, a therapeutic or diagnostic agent and a pharmaceutically acceptable carrier.

21. A pharmaceutical composition comprising a complex according to claim 4 along with a pharmaceutically acceptable carrier.

22. A method of producing a pharmaceutical composition according to claim 21 comprising admixing said complex with one or more pharmaceutically acceptable ingredients.

23. A method of diagnosing a glioblastoma in an individual, said method comprising
a) contacting brain tissue cells with a peptide according to claim 3, wherein said peptide is associated with a diagnostic agent;
b) detecting the presence of the diagnostic agent; and
c) determining the presence of glioma cells.

24. A method according to claim 23 wherein the brain tissue cells have been obtained from a patient.

25. A method of treating an individual having or suspected of having a glioblastoma, said method comprising administering to said individual a peptide according to claim 3 associated with a therapeutic agent.

26. A method according to claim 25 wherein said peptide is administered by oral administration or injection.

27.-29. (canceled)

30. A method of improving the binding affinity of a peptide according to claim 1 comprising modifying the amino acid sequence of the peptide either by addition, deletion or substitution of amino acid residues and then testing said modified peptide for improved binding affinity with glioma cells.

31. A method according to claim 30 wherein the testing step includes the unmodified peptide as a control so as to compare binding affinities.

32. A method of designing a mimetic of a peptide according to claim 1, the mimetic being capable of binding glioma cells, said method comprising
a) analysing said peptide that is capable of binding glioma cells to determine the amino acid residues essential and important for the activity to define a pharmacophore; and
b) modelling the pharmacophore to design and/or screen candidate mimetics having the ability to bind glioma cells.

33. A method according to claim 34 further comprising a step of assaying binding of a candidate mimetic to glioma cells in vitro.
